(19) United States
(12) Patent Application Publication
(10) Pub. No.: US 2023/0093506 A1
(43) Pub. Date: Mar. 23, 2023

(54) UNIT FOR NON-INVASIVE STIMULATION OF THE VISUAL CORTEX CELLS IN SEVERE VISUAL IMPAIRMENT

(71) Applicant: CESKE VYSOKE UCENI TECHNICKE PRAZE, 16000 Praha 6, Dejvice (CZ)

(72) Inventors: Karel HANA, Praha (CZ); Jiri CHOD, Stodulky (CZ); Jan LESTAK, Horni Bezdekov (CZ); Jozef ROSINA, Praha (CZ)

(73) Assignee: CESKE VYSOKE UCENI TECHNICKE PRAZE, 16000 Praha 6, Dejvice (CZ)

(21) Appl. No.: 17/786,640

(22) PCT Filed: Jun. 10, 2021
(86) PCT No.: PCT/IB2021/055086

§ 371 Date: Jun. 17, 2022

(30) Foreign Application Priority Data
Jun. 10, 2020 (CZ) PV 2020-337

(51) Int. Cl. A61N 5/02

(52) U.S. Cl. CPC A61N 5/022

(57) ABSTRACT

The unit for non-invasive stimulation of visual cortex cells in severe visual impairment comprises the image sensor (1) provided with the video chip (2) connected to the control processor (9) to convert the image signal to signals capable of stimulating the brain, to which the first transmitting sensor (3) for self-stimulation, the second transmitting sensor (4) of the position stabilisation and the receiving sensor (8) of the position stabilisation feedback are connected via the position stabilisation unit (6), wherein these sensors are connected to the carrier (16) of the active/passive sensors (7) via transmitting active/passive sensors (7) of the position stabilisation feedback. The communication module is connected to the control processor (9); this communication module may be interconnected to the mobile unit (15), and the individual elements may be placed within the supporting structure (5).

### BACKGROUND AND SUMMARY

The technical solution refers to a unit for non-invasive stimulation of visual cortex cells in severe visual impairment, employing telecommunication technology with electronics and sensors, computer technology and wireless communication; specifically, the solution relates to a unit for non-invasive transcranial stimulation using a specifically modified electronic module and the communication thereof with other devices. Therefore, this is a creation of a “bionic eye” for a person concerned.

The effort to create a “bionic” eye using electronics to substitute sight is a logical outcome of state of the art. It will still represent the only effective way to substitute sight until the genetic manipulation era. The conditions are similar to the at present mastered conditions for hearing compensations; however, these are infinitely more complex regarding the significant specifics of vision, perception, and processing thereof by a human brain.

In principle, this is an electronic image sensor, i.e., a chip, a complete camera, individual sensitive sensors, etc., signal of which is processed by a “video chip”—an analogy of image processing for transmission or display to signals that the brain is capable of receiving. These signals usually represent electrical current modulated in different ways to stimulate the appropriate response. The connection is solved in different ways, either by connecting to the original pathway at the level of the retina, optic nerve or the electrodes are introduced directly into the brain as fine electrodes of various designs. Thus, known embodiments always solve the interconnection using electrodes at the level of subcortical or cortical centres in the brain.

Currently, four bionic eye systems have received marketing approval in Europe and the US, wherein a number of others have undergone preclinical and clinical trials, reflecting the established safety profile for permanent stimulation. This progress demonstrates an effort to help blind patients, hoping for tangible and measurable help.

The microchip with electrodes itself may be implanted intraocularly, i.e., it provides stimulation epiretinally, subretinally, or suprachorioideally.

This represents the most significant complication from the patient's point of view and requires extensive invasive interventions into the eye itself, nerve connections thereof to the brain or directly into the brain, with all possible consequences.

The shortcomings mentioned above are eliminated mainly by the unit for transcranial—non-invasive stimulation of visual cortex cells in severe visual impairment, employing telecommunication technology with electronics and sensors, computer technology, and wireless communication. This invention consists, according to an aspect thereof, in that it comprises an image sensor connected to a video chip to convert the image signal to signals capable of stimulating the brain, and, further, comprises a supporting structure provided with a transmitting sensor, another transmitting sensor for position stabilisation, a position stabilisation unit maintaining the data flow direction to the actual brain cells, maintaining the position stabilisation in cooperation with a sensing sensor for feedback sensor, all of which are controlled by a processor so that the information transmission is always optimal.

The system is complemented by a communication module capable of communicating with a remote monitoring centre using mobile LAN and WAN networks, especially 2G to 5G networks, and, at the same time, providing the possibility of software modifications to the system. Power is included with the unit and is capable of continuous, wireless recharging. If necessary, image data processing can be taken over at the remote centre and sent back to the unit. What is important is the direct transmission of the image information to the brain without internal electrodes implanted in the brain using appropriate frequencies and suitable modulation, resulting in stimulating neurons.

The summary of the invention is precise non-invasive—transcranial stimulation of neurons of the visual brain cortex. The system uses a combination of signals from the entire electromagnetic spectrum, typically frequencies from 5 to 100 GHz, to directly stimulate parts of the brain. The support unit also includes a set of sound and ultrasound wave generators and sensors to help focus the main bundle and support stimulation of the neurons themselves. The stimulation itself is spot focused by an antenna assembly of the transmitting unit. This is placed outside the skull; however, it focuses the section in the brain spottily. The sensing image sensor itself—the image sensor, i.e., the camera—is detachable and can be fitted, for example, into glasses, or it can be part of an extension of the supporting structure, e.g., fitted to a wearer's head, to the position needed to acquire the relevant data.

The exact localisation of the selected neurons can be determined, e.g., by functional magnetic resonance on a visual stimulus as an input calibration or directly during tests of the unit itself.

Visual impairment and blindness remain significant public health problems worldwide. The World Health Organization (WHO) estimates that there are approximately 253 million people with visual impairment worldwide: 36 million were blind, and 217 million had moderate to severe visual impairment in 2015. Given this information, the usage of the proposed device could be huge.

### BRIEF DESCRIPTION OF DRAWINGS

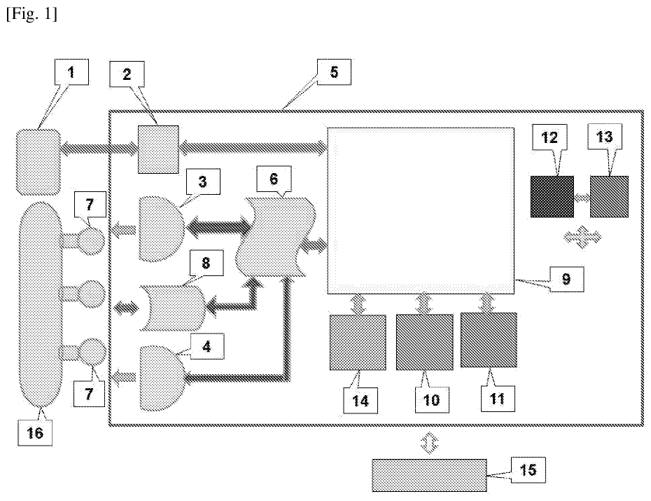

The technical solution is explained in more detail in the attached drawing, wherein FIG. 1 shows an overview diagram of the solution and communication and shows a block diagram of the sensor network, unit modules, and communication thereof.

### DETAILED DESCRIPTION

The example of the unit arrangement for non-invasive stimulation of visual cortex cells according to the present invention is shown as the block diagram in the accompanying drawing FIG. 1. The unit of the image sensor 1 is wired/wirelessly connected to the video chip 2 and then connected to the control processor 9, to which the first transmitting sensor 3 of the antenna assembly and the second transmitting sensor 4 of the position stabilisation are connected via the position stabilisation unit 6, in connection with the receiving sensor 8 of the position stabilisation feedback, connected to a structure of the carrier 16 for the active/passive positioning sensors 7 fixed/implanted on the head of a subject and carrying the active/passive sensors 7 of the position stabilisation feedback. The assembly is completed with the LAN communication module 10 and the WAN communication module 11 along with the GNSS unit 14, all connected to the control processor 9. All of these are powered by a battery, i.e., the power supply unit 12 charged by the rechargeable module 13. All of these are enclosed within the mechanical supporting structure 5, wherein the assembly can also communicate with the mobile unit 15.

The unit for non-invasive stimulation of visual cortex cells in severe visual impairment will find the application thereof for visually impaired people, both blind and moderate to severe visual impairment.

### REFERENCE SIGN LIST

1 Image sensor (camera)
2 Video chip
3 Transmitting sensor A (antennae), self-stimulation
4 Transmitting sensor B, position stabilisation
5 Unit supporting structure
6 Position stabilisation unit (gyroscopic and/or floating)
7 Position stabilisation feedback sensors—passive/active (transmitting)
8 Position stabilisation feedback sensors—receiving
9 Processor
10 LAN communication module
11 WAN communication module
12 Power unit (battery)
13 Rechargeable battery module
14 GNSS unit (Galileo, GLONASS, GPS . . . )
15 Mobile unit (phone, tablet . . . ) to control and set the function
16 Active/passive positioning sensor carrier fixed/implanted on the subject's head

1. A unit for non-invasive stimulation of visual cortex cells in severe visual impairment comprising
an image sensor and
means for stimulating visual cortex cells,
a control processor, the image sensor being provided with a video chip connected to the control processor to convert an image signal to signals capable of stimulating a brain at a frequency of 5 to 100 GHz,
a first transmitting sensor for self-stimulation, a second transmitting sensor of a position stabilisation, and a receiving sensor of a position stabilisation feedback being connected to the control processor via a position stabilisation unit, wherein the image sensor, the first transmitting sensor, the second transmitting sensor, and the receiving sensor are connected to a carrier of active/passive sensors via transmitting active/passive sensors of the position stabilisation feedback, and
a communication module connected to the control processor.

2. The unit according to claim 1, wherein the communication module is selected from the group consisting of a LAN communication module, a WAN communication module, and a GNSS unit, which are interconnected to a mobile unit.

3. The unit according to claim 1, wherein a power supply unit with a rechargeable module is connected to the control processor.

4. The unit according to claim 3, wherein the video chip, the control processor, the position stabilisation unit, the first transmitting sensor, the second transmitting sensor, the receiving sensor, the communication module, and the rechargeable module are placed within a supporting structure.

5. The unit according to claim 2, wherein a power supply unit with a rechargeable module is connected to the control processor.

6. The unit according to claim 2, wherein the video chip, the control processor, the position stabilisation unit, the first transmitting sensor, the second transmitting sensor, the receiving sensor, the communication module are placed within a supporting structure.
